(19) United States
(12) Patent Application Publication
(10) Pub. No.: US 2006/0270913 A1
(43) Pub. Date: Nov. 30, 2006

(54) Surgical console operable to record and playback a surgical procedure

(76) Inventor: Kirk W. Todd, Yorba Linda, CA (US)

Correspondence Address: ALCON, IP LEGAL, TB4-8, 6201 SOUTH FREEWAY, FORT WORTH, TX, 76134, US

(21) Appl. No.: 11/193,216

(22) Filed: Jul. 29, 2005

Related U.S. Application Data
(60) Provisional application No. 60/685,998, filed on May 31, 2005.

(51) Int. Cl. A61B 5/00; A61B 19/00

(52) U.S. Cl. 600/300; 128/898

(57) ABSTRACT

The present invention provides a system and method for programming a surgical console to perform a surgical procedure involving a series of steps. This involves first initializing a procedural recorder wherein the procedural recorder is resident within a surgical console. A first surgical procedure may be performed utilizing the surgical console. During the performance of this first surgical procedure, the procedural recorder records operating parameters and surgical modes associated with the first surgical procedure. Operating parameters and surgical modes associated with the first surgical procedure are recorded or stored as a recorded procedure in memory within the procedural recorder.

This application claims priority under 35 U.S.C. §119 to U.S. Provisional Patent Application No. 60/685,998, filed May 31, 2005, the entire contents of which are incorporated herein by reference.

### TECHNICAL FIELD OF THE INVENTION

The present invention relates generally to surgical consoles systems and methods, and more particularly, a system and method for programming a surgical console to perform a given surgical procedure.

### BACKGROUND OF THE INVENTION

During modern surgery, particularly ophthalmic surgery, the surgeon uses a variety of pneumatic and electronically driven microsurgical handpieces. The handpieces are operated by a microprocessor-driven surgical console that receives inputs from the surgeon or an assistant by a variety of peripheral devices, such as foot pedal controllers, infrared remote control devices and menu-driven touch screens. One such microsurgical console is described in U.S. Pat. No. 5,455,766 (Scheller, et al.), the entire content of which is incorporated herein by reference. Surgical consoles allow surgeons to manually input surgical operating parameters and store these “customized” parameters in the console memory for future use. However, all of these devices require that the operating parameters and methodologies be inputted manually using a keypad, touch screen or downloaded from another console that has had the parameters inputted manually. This manual process is both tedious and error prone.

Accordingly, a need exists for a surgical console that allows for the programming of complex surgical operating parameters and methodologies, without having to input these manually through the use of a keypad, touch screen or external media. This would simplify a tedious and error prone task.

### SUMMARY OF THE INVENTION

The present invention provides a system and method for programming a surgical console operable to control surgical devices during a surgical procedure. This system and method substantially addresses the above-identified needs, as well as others. More specifically, the present invention provides, in a first embodiment, a method of programming a surgical console to perform a surgical procedure involving a series of steps. First, a procedural recorder is initialized wherein the procedural recorder is resident within a surgical console. The surgical procedure may be performed utilizing the surgical console. During the performance of this first surgical procedure, the procedural recorder records operating parameters and surgical modes associated with the first surgical procedure. Operating parameters and surgical modes associated with the first surgical procedure are stored as a recorded procedure in memory within the procedural recorder for future playback during other medical procedures.

Another embodiment provides a surgical console operable to record and execute a recorded surgical procedure. The recorded surgical procedure may be recorded within the surgical console that is executes the recorded procedure at a later time or from a different surgical console. This surgical console may include a microprocessor, memory, procedural recorder, a user interface and interface(s) through which peripheral devices couple to the console. The microprocessor may direct operations of peripheral devices that couple to this surgical console. The memory in addition to containing instructions which the microprocessor uses to direct the operation of the peripheral devices may also store recorded surgical procedures. The user interface allows users or operators to initialize the surgical console for a given surgical procedure, select recorded surgical procedures to be executed for memory, and advance through the steps of the selected surgical procedure. These steps include operating parameters and surgical modes associated with the specific steps. Further, as these steps are selected and loaded into the surgical console, the operating parameters and surgical mode of the surgical console and attached peripheral devices are set. Additionally, due to variations in individual procedures, advances or other like changes in surgical techniques, it may be desirable to store the executed surgical procedure as an additional recorded surgical procedure for future use. This provides significant technical advantages in that operators may effortlessly create or revise procedures for specific disease states and store these to memory without interfering with the current surgical flow. These surgical flows or surgical procedures may be saved after successful application based on the surgical outcome.

The present invention improves upon the prior art by providing a surgical console containing a surgical procedural recording and playback program. Once initiated, the program interprets the surgeon's procedural flow and determines its pertinence to the current surgical state. All pertinent changes to operating parameters and methodologies being used by the surgeon during a surgical procedure are recorded. This recorded procedure may then be used by the surgeon to playback the pertinent operating parameters and procedural steps of the surgical console during similar future surgeries.

The present invention allows the operating parameters of the various surgical devices to be stored in memory and/or downloaded onto media and transferred to other surgical consoles. Such surgical consoles may include, but should not be limited to, the SERIES TWENTY THOUSAND® LEGACY® surgical system and the ACCURUS® surgical system, both available from Alcon Laboratories, Inc. This allows the operator or surgeon to effortlessly create disease process memories without interfering with the surgical flow. The surgical procedures can be saved and edited “after the fact” based upon surgical outcome.

Accordingly, embodiments of the present invention provide a surgical console having a procedural recorder that saves operating parameters into its memory without the tedious and error prone task of manually programming the device through the use of a keypad, a touch screen or external media. These and other advantages and objectives of the present invention will become apparent from the detailed description and claims that follow.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 is a perspective view of one surgical console that may be used with embodiments of the present invention;

FIG. 2 is a flow chart indicating the steps followed by a doctor in recording a surgical procedure as contemplated by an embodiment of the present invention;

FIG. 3 is a flow chart indicating the steps followed by a doctor in playing a recorded surgical procedure as contemplated by an embodiment of the present invention; and

FIGS. 4 through 6 are block diagrams depicting the steps employed by the procedural recorder in accordance with an embodiment of the present invention.

### DETAILED DESCRIPTION OF THE INVENTION

Preferred embodiments of the present invention are illustrated in the FIGUREs, like numerals being used to refer to like and corresponding parts of the various drawings.

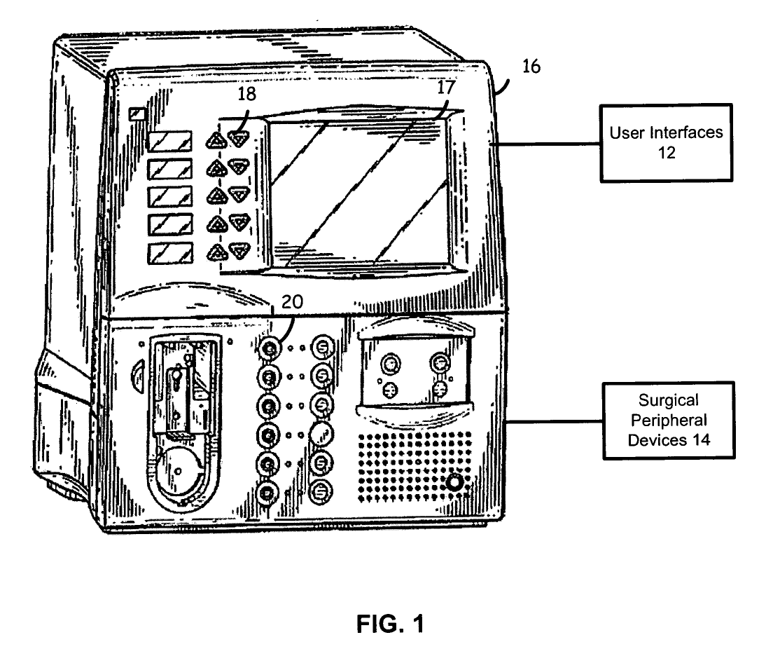

The procedural recorder provided by embodiments of the present invention may be used with any suitable surgical console such as but not limited to the SERIES TWENTY THOUSAND® LEGACY®, the INFINITI® or the ACCURUS® surgical system consoles, as seen in FIG. 1, both commercially available from Alcon Laboratories, Inc., Fort Worth, Tex., that contain programmable memories for storing operating parameters.

FIG. 1 provides an illustration of surgical console 10. Surgical console 10 has user interfaces 12 and may couple to peripheral devices 14, such as a foot pedal assembly or other push-button type assembly not shown. Console 10 allows an operator, such as a surgeon, to begin a surgical procedure by setting the initial operating parameters and modes into the console. This may be done by allowing the operator to interface with the surgical console through user interfaces 12 or other interfaces provided on front panel 16. These may include an electronic display screen 17, a plurality of push-button switches or touch-sensitive pads 18, the plurality of endless digital potentiometer knobs 20, or other like interfaces known to those skilled in the art. Push-button 18 and knobs 20 are actuable by an operator to access various different operating modes and functions used in various surgical parameters. Console 10 may also include the ability to accept storage media such as cassette tapes, memory cards, floppy disks, or other like devices known to those skilled in the art.

Electronic display screen 17 may be controlled by a microprocessor that allows the operator access to one or more different menus or messages which relate to the functions and operations of the various push buttons 18 and knobs 20. In one embodiment, the display screen may be divided into display screen regions associated with individual buttons 18. This arrangement allows for the indicated function of each button 18 or knob to be readily changed. Additionally, the use of the electronic display screen also permits the buttons and knobs to be labeled in virtually any language.

Surgical console 10 may be adapted for use with a number of different surgical instruments (i.e. surgical peripheral devices 14). For example, these may include a fiber optic illumination instrument, a fragmentation emulsification instrument, a cutting instrument, such as a guillotine cutter for vitrectomy procedures, and micro-scissors inset for proportionate and multiple cutting. While the above-identified surgical instruments are provided for illustrative purposes, it should be understood that the console 10 can be used with other similar equipped instruments.

In general, any surgical instruments that are actuated or controlled by pneumatic or electronic signals may be operably coupled to and controlled by console 10. This control or actuation may be governed by pneumatic, electronic, optical, or other like signals known to those skilled in the art wherein the signals are generated by console 10. Each of these illustrated surgical devices that couple to console 10 may have different modes of operation that may require different settings or parameters that are provided by the microsurgical console. By saving these operating parameters and surgical modes which are associated with specific steps of a surgical procedure in memory, the setup of the surgical peripheral devices is facilitated by eliminating the often tedious, cumbersome and error prone process of manually initializing these devices manually via the surgical console for each step of the surgical procedure.

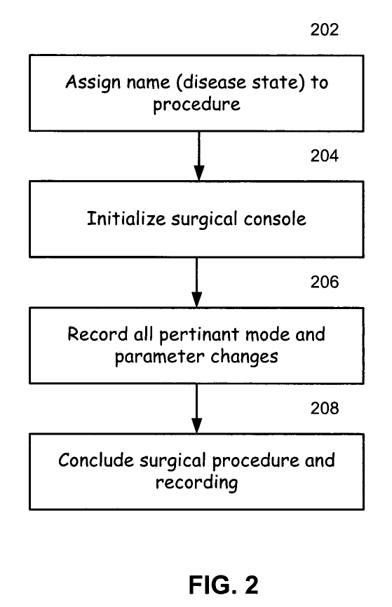

As the operator advances through surgical procedure, pertinent changes to the operating modes and peripheral device operating parameters are accessed from console memory and used to initialize or setup the surgical devices for individual steps within an overall surgical procedure. At the completion of a surgical procedure the completed surgical procedure may be saved as a recorded procedure in memory. It should be noted that within surgical console 10, a microprocessor couples to memory where the microprocessor is operable to execute the steps that will be discussed in the logic flow diagrams of FIG. 2 and following. These steps are accessed by the microprocessor in which the instructions or steps are stored as well as the recorded surgical procedures.

The microprocessor may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The memory may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the microprocessor implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory stores, and the microprocessor executes, operational instructions corresponding to at least some of the steps and/or functions illustrated in FIGS. 2 through 6.

FIG. 2 provides a logic-flow diagram illustrating a methodology associated with an embodiment of the present invention. As seen with reference to FIG. 1, an operator may recall a previously recorded surgical procedure from memory within or coupled to a surgical console such as microsurgical console 10. This surgical procedure is created following the logic-flow diagram provided in FIG. 2. Here, an operator may assign a name to the procedure such that it may be invoked at a later time in step 202. Alternatively, a recently executed procedure may be stored after execution for later recall. In step 204, the operator begins a surgical procedure by initializing the surgical console. Initialization involves setting a number of parameters associated with the microsurgical peripheral devices coupled to the surgical console. The initial parameters and modes associated with the surgical procedure may be stored in memory and retrieved in step 204. As the procedure continues, step 206 records all mode and pertinent parameters changes as the surgical procedure progresses. At the completion of the surgical procedure, the operator may store the current surgical procedure and assign a name, if not already done so, in step 208. This allows those recorded surgical procedures, as executed, to be recalled for future use. Additionally, as the mode and pertinent parameter changes recorded in step 206, may be recorded as individual steps which may be numbered. The operator may edit or name theses procedural steps to facilitate future use of the recorded procedure.

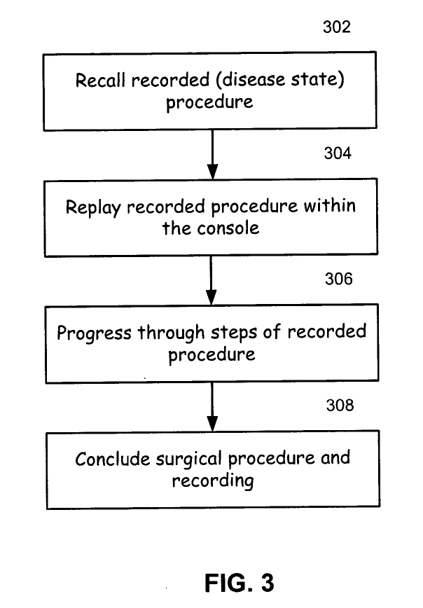

FIG. 3 provides a logic flow diagram illustrating the one method by which a recorded surgical procedure is executed. In step 302, an operator recalls a recorded procedure. This may be done by using a user interface, such as that on surgical console 10, a voice or audio interface, or other like means known to those having skill in the art. One embodiment may allow the operator to recall the recorded procedure by specifically pressing a touch-button (one touch button) associated with the name of the surgical procedure to be utilized. This surgical procedure may be named by the disease state to be addressed. The console plays the retrieved surgical procedure in step 304. As the procedure is replayed, the surgical console adjusts the operating parameters and modes of the peripheral devices. In step 306, the operator progresses through the steps of the recorded procedure. This may be done using a user interface, such as a voice switch, to confirm with prompts and inform the operator of changes associated with selected procedural steps. The operator proceeds through all the recorded steps until completion.

Existing surgical consoles may have the ability to play back recorded surgical procedures but may not necessarily the ability to record surgical procedures. Embodiments may be implemented by software changes within existing surgical consoles to implement embodiments of the present invention rather than hardware changes.

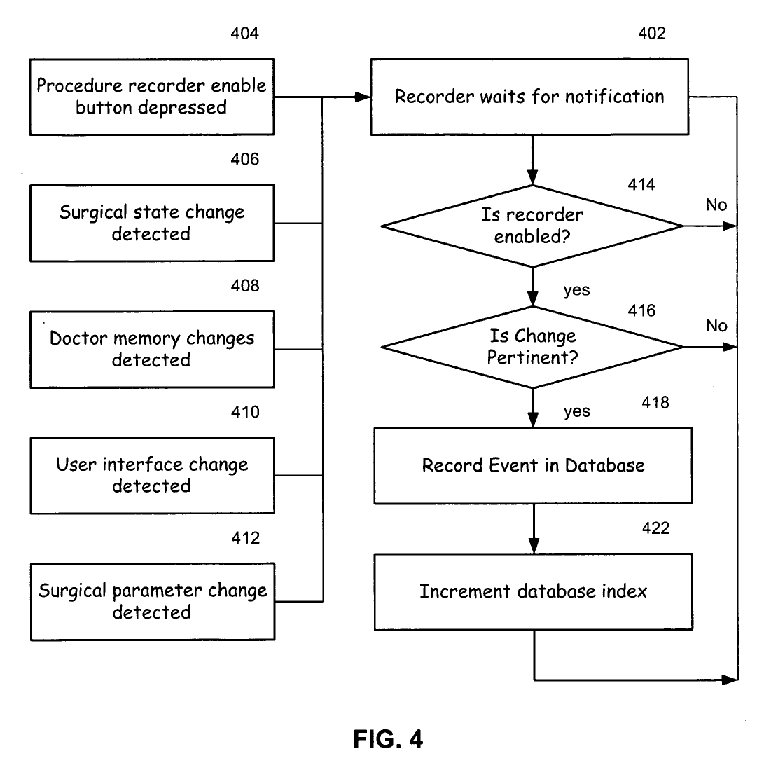

FIGS. 4, 5 and 6 illustrate the logic employed by specific embodiments of a procedural recorder as provided by present invention. The recorded procedure may be used for to perform additional surgical procedures by recalling the recorded surgical procedure from memory attached to the console to initialize, reinitialize or alter the mode or operating parameters associated with peripheral devices as the surgical procedure progresses. This is achieved by allowing the recorded procedure to automatically adjust the settings and operating parameters associated with the console as the surgical procedure progresses. A user may scroll through various steps in the recorded procedure through the use of a console foot switch, remote control, touch screen, voice commands, or other like means known to those skilled in the art

As seen in FIG. 2, the procedural recorder of the present invention initializes the surgical equipment. The surgeon then begins the surgical procedure. Initial operating parameters and modes are saved to memory by the recorder for future review, if needed. As the surgeon continues through the surgical procedure, pertinent changes in the operating modes and peripheral device operating parameters are saved by the recorder to memory. When the surgical procedure is completed, the recorded procedure is saved to memory and assigned a name, such as a Disease State, to the procedure to be recorded and the recorder is turned off.

As seen in FIG. 3, when the operator desires to use a previously recorded procedure, the operator recalls the recorded procedure from the console memory and initializes the equipment. The recorded procedure then automatically adjusts the settings and operating parameters of the console as required by the surgical flow. Scrolling through the various steps in the procedure can be accomplished by use of the console footswitch, remote control or console touch screen. The commands may be confirmed via voice confirmation by the console.

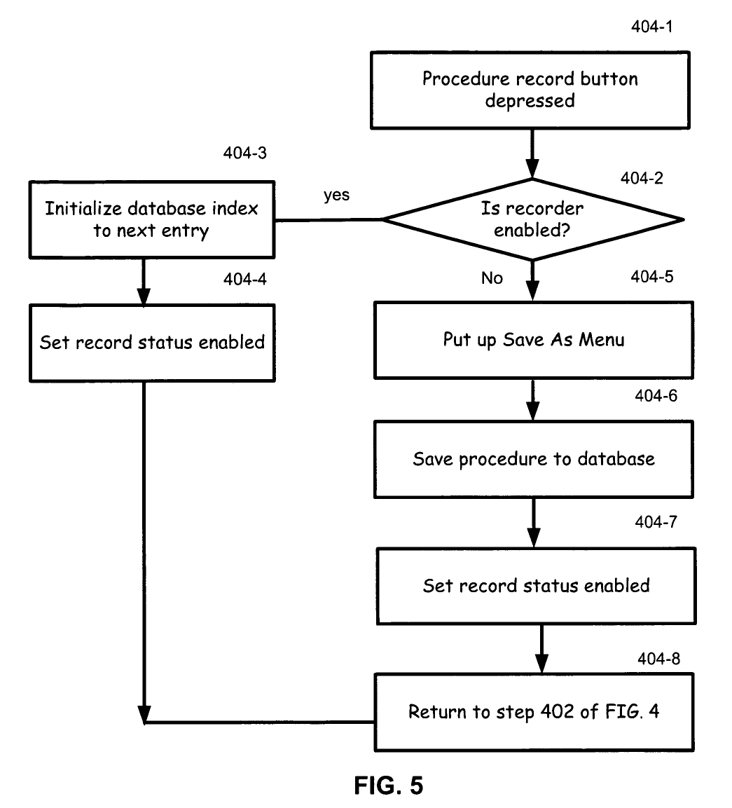

FIG. 4 provides a logic flow diagram associated with the methodology of creating a recorded surgical procedure. Beginning with step 402, a procedural recorder may await notification of status changes associated with operating parameters or modes of operation of the microsurgical peripheral devices or console. These may include “enabling the procedure recorder button” in step 404 which will be discussed further with reference to FIG. 5; a change in a surgical state as detected by the surgical console and logic executed therein in step 406; changes in operator memory as detected by the operator's database in step 408; a change as detected through the user interface in step 410; and/or the change of any surgical parameter as detected by sensors associated with each surgical devices in step 412. Upon detecting notification of any of these changes in operating modes or parameters, the procedural recorder upon notification will determine whether or not the procedural recorder is enabled at decision point 414. If the procedural recorder is not enabled the procedural recorder may return to a waiting state as provided in step 402. Otherwise, when the procedural recorder is enabled at decision point 414 decision point 416 will determine whether or not the change reported to the procedural recorder in step 402 is pertinent to the surgical procedure. If the change is determined to not be pertinent at decision point 414, the process will return to step 402 and wait for the notification of any additional changes. Otherwise, step 418 will record the event in procedural recorder database 420 and then increment the database index at step 422. After which the process will return to step 402 and await another notification of change to a mode of operation or operating parameter associated with the surgical procedure.

Returning to step 404, FIG. 5 provides a logic flow diagram that further details the processes associated with step 404. More specifically, step 404-1 determines whether or not the procedural recorder is activated. This may involve the depression of a record button or other like function. Then at decision point 404-2, a determination is made as to whether or not the record mode is enabled. If the record mode is enabled, step 404-3 will initialize the database index to the next available entry and then step 404-4 will set the record status to enable. Otherwise, if the record mode is not enabled at step 404-2, the user interface on the console may display in step 404-5 a “save as” procedure prompt. This allows the operator to save the current procedure under a new or existing procedure name in step 404-6. In step 404-6, the procedure is saved to the procedural recorder database and the record status is set to enable in step 404-7. Step 404-8 returns to the logic flow diagram of FIG. 4 at step 402.

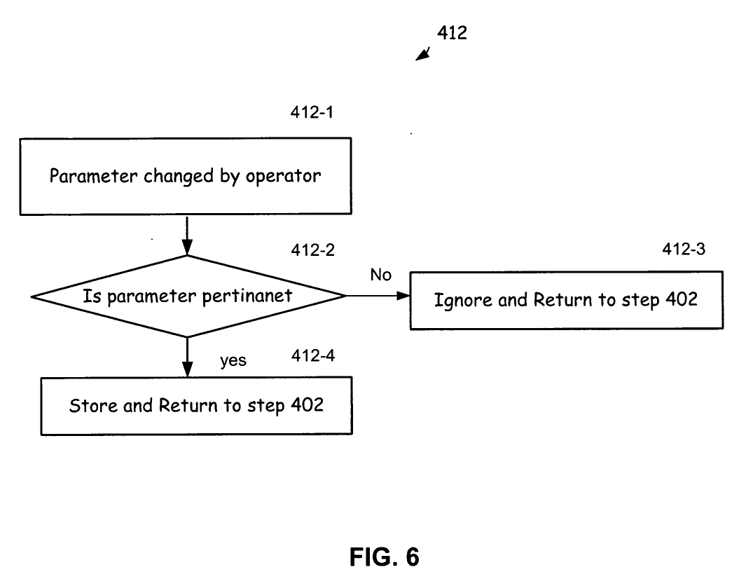

FIG. 6 determines whether or not the parameter has been changed by the operator as previously described with respect to step 412 of FIG. 4. In step 412-1, a parameter is changed by the operator. Decision point 412-2 determines whether the parameter is pertinent to the current surgical state. If is the change is pertinent, this information is supplied to a procedural recorder in step 412-4. This process then returns to step 402 of FIG. 4. Otherwise, a decision is made to ignore parameters not pertinent to the current surgical state at step 412-3 and the process then returns to step 402 of FIG. 4.

In summary, the present invention provides a system and method for programming a surgical console to perform a surgical procedure involving a series of steps. This involves first initializing a procedural recorder wherein the procedural recorder is resident within a surgical console. A first surgical procedure may be performed utilizing the surgical console. During the performance of this first surgical procedure, the procedural recorder records operating parameters and surgical modes associated with the first surgical procedure. Operating parameters and surgical modes associated with the first surgical procedure are recorded or stored as a recorded procedure in memory within the procedural recorder.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”.

As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

The present invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims.

What is claimed is:

1. A method of programming a surgical console to perform a surgical procedure, comprising:
initializing a procedural recorder wherein the procedural recorder being resident in the surgical console;
performing a first surgical procedure with the surgical console;
recording surgical console operating parameters and surgical modes associated with the first surgical procedure with the procedural recorder; and
storing the surgical console operating parameters and surgical modes associated with the first surgical procedure as a recorded procedure wherein the recorded procedure is stored in memory.

2. The method of claim 1, further comprising:
retrieving the recorded procedure from memory; and
utilizing the recorded procedure to perform a second surgical procedure.

3. The method of claim 2, wherein the operating parameters and surgical modes associated with the surgical procedure(s) comprise a series of surgical steps operable to:
alter operating modes of devices operably coupled to the surgical console; or
alter peripheral device operating parameters of devices operably coupled to the surgical console.

4. The method of claim 3, wherein a user interface allows an operator to advance through the recorded procedure, when the recorded procedure is replayed.

5. The method of claim 2, wherein the surgical procedure is ophthalmic surgical procedure.

6. The method of claim 2, wherein the operating parameters and surgical modes associated with the surgical procedure(s) comprise:
pneumatic and electronic parameters associated with microsurgical peripheral devices operably coupled to the surgical console.

7. The method of claim 2, wherein the recorded procedure is operable to be played back within a second surgical console.

8. A surgical console operable to execute a recorded surgical procedure, comprising:
a microprocessor operable to direct operations of peripheral devices operable coupled to the surgical console;
a memory operable coupled to the microprocessor, wherein the memory is operable to store recorded surgical procedures; and
a user interface, wherein the user interface allows operators to:
initialize the surgical console for a surgical procedure;
select the recorded surgical procedure to be executed;
advance through the steps of the selected surgical procedure, wherein operating parameters and surgical modes associated with specific steps of the selected recorded surgical procedure are loaded to the surgical console; and
store surgical procedures as additional recorded surgical procedures.

9. The surgical console of claim 8, wherein the operating parameters and surgical modes associated with the surgical procedure(s) comprise a series of surgical steps operable to:
alter operating modes of devices operably coupled to the surgical console; or
alter peripheral device operating parameters of devices operably coupled to the surgical console.

10. The surgical console of claim 8, wherein the surgical procedure is ophthalmic surgical procedure.

11. The surgical console of claim 8, wherein the operating parameters and surgical modes associated with the surgical procedure(s) comprise:
pneumatic and electronic parameters associated with microsurgical peripheral devices operably coupled to the surgical console.

12. The surgical console of claim 8, wherein the recorded procedure is operable to be played back within a second surgical console.
